(19) United States
(12) Patent Application Publication
(10) Pub. No.: US 2010/0178315 A1
(43) Pub. Date: Jul. 15, 2010

(54) POLYMERIC SYSTEMS FOR CONTROLLED DRUG THERAPY

(75) Inventors: Jeanne Y. Ellis, Andover, MA (US); Edward J. Ellis, Andover, MA (US)

Correspondence Address: Leason Ellis LLP, 81 Main Street, Suite 503, White Plains, NY, 10601, US

(73) Assignee: Vista Scientific LLC, Andover, MA (US)

(21) Appl. No.: 12/633,093

(22) Filed: Dec. 8, 2009

Related U.S. Application Data
(62) Division of application No. 11/359,156, filed on Feb. 22, 2006.
Application No. 11/359,156 is a continuation of application No. PCT/US2004/026775, filed on Aug. 19, 2004.
(60) Provisional application No. 60/497,298, filed on Aug. 22, 2003.

(51) Int. Cl. A61K 31/5377; A61F 2/00; A61P 27/06

(52) U.S. Cl. 424/427; 514/236.2

(57) ABSTRACT

Polymeric compositions containing a high percentage of bound alkyl ether segments provide matrices and membranes for the controlled release of drugs and medicinal agents. The polymeric compositions are prepared by the polymerization of ethylenically unsaturated alkyl ether containing monomers. Copolymers of ethylenically unsaturated alkyl ether containing monomers with co-monomers are also disclosed. The drug loaded polymeric compositions of this invention find particular utility in the construction of controlled release devices.

### CROSS REFERENCE TO RELATED APPLICATIONS

This application is related to and claims the benefit of International Application No. PCT/US2004.026775 filed on Aug. 19, 2004 which claims priority to U.S. Provisional Patent Application No. 60/497,298 filed on Aug. 22, 2003, both of which said applications are herein incorporated by reference in their entirety.

### STATEMENT REGARDING FEDERAL SPONSORSHIP

The U.S. Government has a paid-up license in the invention and the right in limited circumstances to require the patent owner to license others on reasonable terms as provided for by the terms of Grant No. 2R44EY13479-02 awarded by the National Institute of Health.

### TECHNICAL FIELD

This invention relates to a polymeric composition, a method and a device for the controlled administration of therapeutically active agents. More particularly, this invention relates to polymeric compositions, which are tailored to impart prescribed release characteristics to a drug dispensing device. In a preferred embodiment, the invention relates to device and system for the controlled and continuous administration of a drug to a mammalian patient over a prolonged period of time. Another aspect of this invention relates to a method of preparing these devices.

### BACKGROUND

Controlled release technology emerged from the 1960's with the promise to solve a diversity of problems that have in common the application of some active agent to a system with the objective of accomplishing a specific purpose while avoiding certain other possible responses this agent might cause. A number of techniques for effecting controlled release have been identified and analyzed, and most of these have been considered for or embodied in commercial devices or formulations, which already are, or soon will be, on the market. Most of the concepts for controlled release of an active agent have been described in the literature, such as patents, journals, books and symposia proceedings.

Controlled release technology has been considered for a wide variety of applications, of which a large fraction are either medically related or for pest control. One of the central problems in controlled release formulations is to combine the active agent with its carrier in an economical manner, yet achieve a release profile that best fits the situation. These two desires are often in opposition to one another, so compromises must be made. In some cases the desired release profile is a constant rate of delivery of the active agent, which, in analogy with chemical kinetics, has become known as a “zero order” process, since it does not depend on how much of the active agent has been delivered or remains. However, many of the devices and formulations used in controlled release technology do not meet this objective.

Polymers and active agents have been closely linked since the beginnings of drug delivery research as evidenced by the progress that has been made in orally administered drugs. Polymers, both synthetic and natural, have been utilized to control the release of orally administered drugs in the gastrointestinal tract. These medications are often taken as pills, tablets or capsules. The polymers utilized in orally administered medications are generally either water soluble or biodegradable.

On the other hand, with few exceptions, the classical approach to drug delivery of non-orally administered medications was to load the drug of choice into common polymeric matrices, such as polyhydroxyethylmethacrylate hydrogels, silicones and ethylene vinyl acetate, to name a few. From release rate studies one of the standard polymer systems was selected to provide performance characteristics that was the closest to ideal. While this approach is pragmatic, it quite often does not produce optimum results. However, over the years a number of controlled release drug delivery devices have been commercialized. Wu has listed some of theses devices and these are presented below in Table I.

TABLE I
Some commercially available controlled-release drug delivery devices
(Xue Shen Wu, “Controlled Drug Delivery Systems”,
Technomic Publishing Co., Inc, 1996)

| Drug | Trade Name | Type of Device | Manufacturer |
|---|---|---|---|
| Scopolamine | Transderm-Scop | Transdermal | Alza/Ciba-Geigy |
| Nitroglycerin | Transderm-Nitro | Transdermal | Alza/Ciba-Geigy |
| | Deponit | Transdermal | Pharma-Schwarz |
| Pilocarpine | Ocusert | Implant | Alza |
| Progesterone | Progestsert | IUD | Alza |
| Levonorgestrel | Norplant | Implant | Population Council |
| Phenyl- | Acutrim | Oral osmotic | Alza-Ciba |
| propanolamine | | pump | |
| LHRH | Lupron Depot | Injectable | TAP Pharm. |
| | Decapeptyl | microspheres | Ipsen Biotech |

LHRH: luteinizing hormone releasing hormone
IUD: intrauterine device

Controlled release systems can be simply classified into physical or physicochemical systems or biochemical systems, according to release mechanisms of the active agent.

Physical or Physicochemical Systems

The physical or physicochemical systems include reservoir systems, matrix or monolithic systems, swelling-controlled systems or hydrogels, osmotic systems or osmotic pumps, transdermal systems and liposomal systems.

Chemical or Biochemical Systems

Biodegradable polymer systems—this category includes biodegradable polymeric systems and bioadhesive systems.

In physiochemical systems, drug release is controlled entirely by physiochemical processes such as diffusion, osmosis, dissolution, etc. The drugs may either be contained within a polymeric membrane or immobilized membrane or dissolved/dispersed homogeneously throughout a polymer or other carrier material, exhibit a release which is controlled by the diffusion of the drug through the carrier material and/or the dissolution of the carrier. Drug release can be activated by the osmotic pressure generated by the active ingredient that controls the diffusion of solvent into the dosage form matrix.

A monolithic matrix is the simplest and least expensive system used to control the drug delivery. The fabrication processes for these systems are similar to those for conventional dosage forms and are highly reproducible. The polymer or other carrier material is homogeneously distributed with the drug by blending the drug with the polymer material and then molding, extruding, or casting them together. The interstices of the polymeric material control the drug release. The degree of diffusion control of the drug within the matrix is determined by the properties of the polymer and the drug. Ideally, drug can exist in one of two states within the polymer matrix. Either the drug is completely dissolved in the polymer, or is purely dispersed as discrete solid drug particles within the polymer matrix. The latter condition prevails when the drug concentration is much higher than the drug's solubility in the solubility in the polymer. In the former condition, the drug is dissolved at or below its solubility in the polymer. The release kinetics of the drug from two states is different. Generally, polymers used for this application either do not respond to changes in the surrounding environment or are rubbery state polymers. A polymer in the rubbery state responds to and adjusts to changes in its environment very rapidly, and the diffusion process of any substance within polymer matrix is Fickian. In addition to diffusion of the drug from the polymer matrix, other physiochemical properties of the polymer may influence the release kinetics. Release characteristics from monolithic matrix systems depend on the nature of the polymer, the additives, the drug, and the geometry of the system. Controlling the release kinetics of a monolithic matrix system is easier than for other systems, i.e. coated systems.

### SUMMARY

The preparation of polymeric products for use in animals and humans is provided herein. More particularly, it is concerned with polymeric membranes, matrices or carriers in the form of a device that regulates the release of drug or active agent in a controlled and prescribed manner. Specifically, it is concerned with devices and components containing therapeutically active agents, which can be used in the treatment of medical diseases or disorders.

Surprisingly, polymeric compositions containing a high proportion of alkyl ether groups have been found to have utility in the construction of devices that provide controlled release of a wide range of drugs over a prolonged period of time. This provides a number of advantages not found in current drug delivery systems.

The alkyl ether polymers of this invention can be utilized in a number of controlled drug delivery devices which include; reservoir systems, matrix or monolithic systems, swelling controlled systems, osmotic systems or osmotic pumps and transdermal systems.

Accordingly, one object is to provide a device for the administration of a locally or systemically acting agent to produce a physiologic or pharmacologic effect which also provides technological advancement over prior art devices.

Another object is to provide a dosage regimen for administering an active agent to a target area for a particular time period, the use of which requires intervention only for initiation and termination of the regimen.

Further, another object is to provide a device for delivering drug that is in the form of a transdermal patch, an osmotic pump, an ocular insert and ocular implants.

Yet another object is to describe processes for making such drug dispensing devices having enhanced mechanical and physical properties.

The compositions of this invention find particular utility, when formed into an ocular drug delivery device, in the treatment of a wide variety of ocular disorders and diseases such as infection, inflammation, glaucoma, diabetic macular edema and age related macular degeneration.

### BRIEF DESCRIPTION OF THE DRAWING FIGURES

FIG. 1 is a plot of cumulative weight, in micrograms, of Timolol drug release versus time;

FIG. 2 is a plot of cumulative weight, in micrograms, of Timolol drug release versus time; and

FIG. 3 is a plot of cumulative weight, in micrograms, of Timolol drug release versus time.

### DETAILED DESCRIPTION OF PREFERRED EMBODIMENTS

In accordance with the practice of presently disclosed devices, it has now been unexpectedly found that certain polymeric materials can be used for forming devices for the controlled release of an active agent, such as a pharmaceutical composition (e.g., a drug), for example by diffusion. As used throughout the present application, the term “medicinal agent” or “drug” refers to any number of types of active agents in a number of different forms, such as a pharmaceutical drug.

The use of and advantages realized by the disclosed polymeric materials are unexpected because they can be formulated to accept high levels of drug loading and exhibit release over a prolonged period of time. Furthermore, polymeric materials can be formulated to accommodate a wide variety of drugs, both hydrophilic and hydrophobic types. The present polymeric materials are compatible with human tissue. That is, these materials do not break down in situ, there is no absorption of the materials, and there is no deleterious action on the sensitive tissues in the area of placement and retention of the system over a prolonged period of time.

The polymers suitable for the purpose of any of the exemplary devices disclosed herein include polymers, copolymers and the like, that are prepared and formed into desired shapes by casting, molding, extrusion or other fabrication processes known in the art.

According to one exemplary embodiment, polymeric materials are disclosed that are suitable as matrices or membranes for the controlled delivery of drugs. The polymeric material that forms the polymeric matrix or membrane comprise alkyl ether segments having the formula:

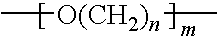

where n=2 to about 10

and m=1 to about 50

The alkyl ether segment contains at least one ethylenically unsaturated moiety that can enter into a polymerization reaction and generally has the following structure:

P-Y-

where: P is an ethylenically unsaturated polymerizable group chosen from among

CH₂═CH— or

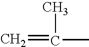

and Y is a spacer group chosen from, but not limited to:
—CO—
—OCO—
—CONHCH₂—
—CONHCH₂CH₂CH₂—
—COOCH₂CH₂NHCOCH₂—
—COOCH₂CH₂NHCH₂CH(OH)CH₂—
—CH₂—
—CH₂CH₂—
—CH₂CH₂CH₂—
—CH₂CH₂CH₂CH₂—
—C₆H₄—
—C₆H₄CH₂—
—COOCH₂CH(OH)CH₂—
—COOCH₂CH₂—
—COOCH₂CH₂OCH₂CH₂— and
—COOCH₂CH₂NHCO—

Examples of ethylenically unsaturated alkyl ether compositions include, but are not limited to:

P-Y-O—(CH₂)ₓ—[O—(CH₂)_{y}]ₙ—O-T

where: P is an ethylenically unsaturated polymerizable group;
Y is a spacer group;
T is a terminal group, which is an alkyl group or a P-Y group
x is an integer from 2 to about 6
y is an integer from 2 to about 8
n is an integer 0 to about 50

Exemplary alkyl ether containing monomers that are suitable for use in the present compositions include:

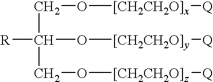

where: Q is independently an alkyl group or P-Y-;
P is an ethylenically unsaturated polymerizable group;
Y is a spacer group;
R is hydrogen or alkyl;
and at least one Q group is P-Y- and x, y and z are independently integers from 1 to about 50; or

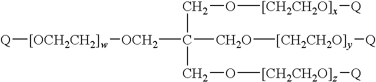

where: Q is independently an alkyl group or P-Y-;
P is an ethylenically unsaturated polymerizable group;
Y is a spacer group;
w, x, y and z are independently integers from 1 to about 50;
and at least one Q group is P-Y-; or

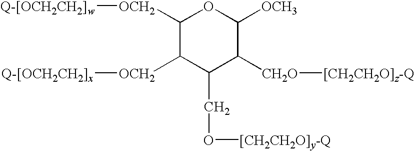

where: Q is independently an alkyl group or P-Y-;
P is an ethylenically unsaturated polymerizable group;
Y is a spacer group;
w, x, y and z are independently integers from 1 to about 50;
and at least one Q group is P-Y-; or

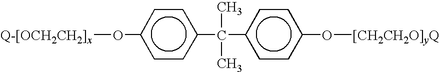

where: Q is independently an alkyl group or P-Y-;
P is an ethylenically unsaturated polymerizable group;
Y is a spacer group;
x and y are independently integers from 1 to about 50;
and at least one Q group is P-Y-.

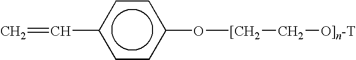

where: T is a terminal group, which is an alkyl group;
n is an integer from 1 to about 50; or

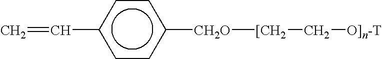

where: T is a terminal group, which is an alkyl group;
n is an integer from 1 to about 50; or

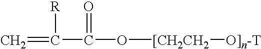

where: R is hydrogen or methyl;
T is a terminal group, which is an alkyl group;
n is an integer from 1 to about 50; or

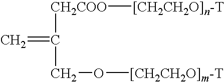

where: T is a terminal group, which is an alkyl group;
n and m are independently integers from 1 to about 50; or

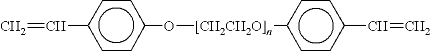

where: n is an integer from 1 to about 50; or

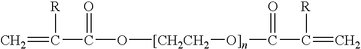

where: R is hydrogen or methyl; and
n is an integer from 1 to about 50.

According to one embodiment, preferred alkyl ether containing monomers include:

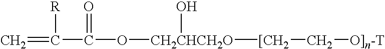

where: R is hydrogen or methyl;
T is a terminal group, which is an alkyl group;
n is an integer from 1 to about 50; or

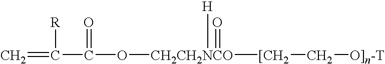

where: R is hydrogen or methyl;
T is a terminal group, which is an alkyl group;
n is an integer from 1 to about 50; or

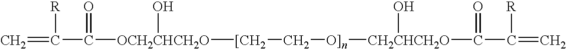

where: R is hydrogen or methyl;
n is an integer from 1 to about 50; or

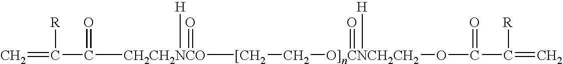

where: R is hydrogen or methyl;
n is an integer from 1 to about 50.

More preferred alkyl ether containing monomers include:

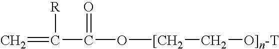

where: R is hydrogen or methyl;
T is a terminal group, which is an alkyl group;
n is an integer from 1 to about 50; or

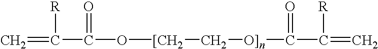

where: R is hydrogen or methyl; and
n is an integer from 1 to about 50.

Most preferred alkyl ether containing monomers include:
Methoxy ethyl acrylate and methacrylate
Methoxy propyl acrylate and methacrylate
Methoxy butyl acrylate and methacrylate
Methoxy ethoxy ethyl acrylate and methacrylate
Ethoxy ethyl acrylate and methacrylate
Ethoxy ethoxy ethyl acrylate and methacrylate
Triethylene glycol monomethyl ether acrylate and methacrylate
Di(ethylene glycol)2-ethylhexyl ether acrylate and methacrylate
Ethylene glycol diacrylate and dimethacrylate
Diethylene glycol diacrylate and dimethacrylate
Triethylene glycol diacrylate and dimethacrylate
Tetraethylene glycol diacrylate and dimethacrylate
Polyethylene glycol diacrylate and dimethacrylate
1,4 butanediol diacrylate and dimethacrylate
Di(1,4 butanediol) diacrylate and dimethacrylate
Tri(1,4 butanediol) diacrylate and dimethacrylate
Tetra(1,4 butanediol) diacrylate and dimethacrylate
Poly(1,4 butanediol) diacrylate and dimethacrylate

Also of use are macromers prepared from polyalkylether diols. The diol is reacted with 2 mole equivalents of a diisocyanate such as diisophorone diisocyanate or toluene diisocyanate. This prepolymer is end-capped with an ethylenically reactive group. The vinylic reactive macromers described here are useful in the practice of this invention.

In preparing the polymeric matrices and membranes, it is often preferable to form copolymers of the alkyl ether containing monomer with one or more comonomers. The drug release profile from these copolymer matrices or membranes can be altered considerably by the choice of comonomer(s). For example, use of a hydrophobic comonomer(s) with the alkyl ether containing monomer will form matrices or membranes that will be compatible with drugs that are hydrophobic. On the other hand, use of a hydrophilic comonomer(s) will produce matrices and membranes that are more compatible with hydrophilic drugs. The release profile of a drug from matrices or membranes described in this invention can also be altered by the degree of crosslinking. Matrices or membranes with higher degrees of crosslinking will retard the diffusion of the drug from the matrix or membrane, thus providing slower release rates.

The monomers, which can be present in the polymers used to form a drug release device, can be any copolymerizable vinyl monomer. The following are representative groups of comonomers that can be employed and serve as examples only and are not intended to limit the scope of the invention.

Suitable comonomers include alkyl acrylates and methacrylates, especially C₁-C₂₀ alkyl acrylates and C₁-C₂₀ alkyl methacrylates, such as methyl methacrylate, ethyl methacrylate, methyl acrylate, butyl methacrylate, butyl acrylate, 2-ethylhexyl acrylate, 2-ethylhexyl methacrylate and the like; alkonoic vinyl esters, especially C₁-C₆alkanoic vinyl esters such as vinyl acetate, vinyl butyrate and the like; alkenes, especially C₁-C₈ alkenes, including ethylene, 1-butene, 1-hexene, and the like; styrenes, especially styrene and alpha-methyl styrene; vinyl ethers, especially C₁-C₆ alkyl vinyl ethers, including methyl vinyl ether, ethyl vinyl ether and butyl vinyl ether, and the like; dialkyl maleates, fumarates or itaconates, especially C₁-C₆ dialkyl maleates, fumarates or itaconates, including dimethyl maleate, dimethyl fumarate, diethyl maleate, dimethyl itaconate and the like; allyl ethers and esters, especially allyl C₁-C₆ alkyl ethers and allyl C₂-C₆ alkanoate esters, including allyl methyl ether, allyl ethyl ether, allyl acetate and the like; perfluoro C₃-C₆ alkyl acrylates or methacrylates; perfluoroalkoxylated bis-acrylates or -methacrylates; poly- or oligo-alkylsiloxane acrylates or methacrylates, and the like.

Also, minor amounts of a crosslinking agent, to alter drug release characteristics, stability and the mechanical properties of the polymer are generally employed. Suitable crosslinking agents include, for example, C₂-C₆ alkylene, di-methacrylates and acrylates, glycerine trimethacrylate; allyl acrylate or methacrylate, divinyl benzene, poly- or oligo-alkylsiloxane di-acrylate or -methacrylate, and the like.

Suitable hydrophilic comonomers are hydroxyl-substituted lower alkyl acrylates and methacrylates, acrylamide, methacrylamide, (lower alkyl)acrylamides and -methacrylamides, N,N-dialkyl-acrylamides, ethoxylated acrylates and methacrylates, polyethyleneglycol-mono(meth)acrylates and polyethyleneglycolmonomethylether-(meth)acrylates, hydroxyl-substituted (lower alkyl)acrylamides and -methacrylamides, hydroxyl-substituted lower alkyl vinyl ethers, sodium vinylsulfonate, sodium styrenesulfonate, 2-acrylamido-2-methylpropanesulfonic acid, N-vinylpyrrole, N-vinyl-2-pyrrolidone, 2-vinyloxazoline, 2-vinyl-4,4′-dialkyloxazolin-5-one, 2- and 4-vinylpyridine, amino(lower alkyl)- (where the term “amino” also includes quaternary ammonium), mono(lower alkylamino)(lower alkyl) and di(lower alkylamino)(lower alkyl)acrylates and methacrylates, allyl alcohol and the like. Preference is given for example, to N-vinyl-2-pyrrolidone, acrylamide, dimethyl acrylamide, methacrylamide, 2-(dimethylamino)ethyl acrylate and methacrylate, 3-(dimethylamino)propyl acrylate and methacrylate, 2-(diethylamino)ethyl methacrylate and methacrylate, 3-(dimethylamino)propyl acrylamide and methacrylamide, hydroxyl-substituted lower alkyl acrylates and methacrylates, hydroxy-substituted (lower alkyl)acrylamides and -methacrylamides and vinylically unsaturated carboxylic acids having a total of 3 to 5 carbon atoms, particularly acrylic and methacrylic acid.

Suitable fluorinated monomers include 1,1,2,2-tetrahydroperfluorodecyl acrylates and methacrylates, 1,1,2,2-tetrahydroperfluorooctyl acrylate and methacrylate and 1,1,2,2-tetrahydroperfluorooctyl methacrylamide or acrylamide, 2,2,2-trifluoroethyl acrylate and methacrylate, hexafluoroisopropyl acrylate, hexafluoroisopropyl methacrylate, perfluorocylcohexyl methacrylate, and 2,3,4,5,6-pentafluoro-styrene; the acrylates and methacrylates of fluoroalkyl substituted amido-alcohols, such as of C₇F₁₅CON(C₂H₅)C₂H₄OH; of sulfonamido-alcohols, such as of C₈F₁₇C₈H₄SO₂N(CH₃)—C₄H₈OH and C₈C₁₇SO₂N(C₂H₅)—C₂H₄OH; of perfluoroether alcohols, such as of C₃F₇—O(C₃F₆O)₂CF(CF₃)—CH₂OH or (CF₃)₂CFO(CF₂CF₂)₂—CH₂CH₂OH; and the acrylates and methacrylate of fluorinated thioether alcohols of structure CF₃(CF₂)_{∫}CH₂CH₂SCH₂CH₂CH₂OH; acrylates and methacrylates of sulfonamido-amines, such as of R_{∫}SO₂NH(CH₃)CH₂CH₂N(CH₃)—(CH₂)₃NH and R_{∫}CH₃SO₂NH(CH₂)₂; of amido-amines, such as of R_{∫}CONH(CH₂)₂NH₂; as well as the vinyl monomers obtained by reaction of these aforementioned fluorinated alcohols and amines with 2-isocyanatoethyl acrylate or methacrylate or m-isopropenyl-1,1-dimethylbenzyl isocyanate.

Suitable silicone containing vinyl monomers are oligosiloxanyl-silylalkyl acrylates and methacrylates containing from 2-10 Si-atoms. Typical representatives include: tris(trimethylsiloxy-silyl)propyl(meth)acrylate, triphenyldimethyl-disiloxanylmethyl(meth)acrylate, pentamethyl-disiloxanylmethyl(meth)acrylate, tertbutyl-tetramethyl-disiloxanylethyl(meth)acrylate, methyl-di(trimethylsiloxy)silylpropyl-glyceryl(meth)acrylate; pentamethyldi-siloxanyl-methyl methacrylate; heptamethyl-cyclotetrasiloxy methyl methacrylate; heptamethyl-cyclotetrasiloxy-propyl methacrylate; (trimethylsilyl)-decamethyl-pentasiloxy-propyl methacrylate; dodecamethyl pentasiloxypropyl methacrylate.

While copolymerization is a preferred means of tailoring the resulting polymer to provide controlled diffusion of an active agent the use of plasticizers can also be employed. Incorporation of a plasticizer into the polymeric matrices or membranes of this invention will alter the diffusion characteristics of the active agent. The incorporation of plasticizers into a polymeric matrix or membrane will result in increased diffusion rate of the active agent. The use of plasticizers will also result in altered mechanical properties of the polymeric matrix or membrane. Representative classes of plasticizers that can be employed in the practice of this invention include, but are not limited to; adipates, citrates, maleates, phthalates and trimellitates.

In certain applications of drug delivery, namely transdermal delivery, penetration enhancers may be utilized. The penetration enhancers loosen the cell structure of tissue, such as the skin, to allow the active agent to diffuse into the tissue structure more easily. Representative classes of penetration enhancers that can be employed in the practice of this invention include, but are not limited to; sulfoxides, acetamides, formamides, toluamides, pyrrolidones, and higher saturated and unsaturated carboxylic acids. The higher carboxylic acids are of particular interest since they will form an acid/base pair with amine containing drugs such as timolol. As an example, heptanoic acid, oetanoic acid, lauric acid, 2-ethylhexanoic acid, sorbic acid and elaidic acid are useful in this function.

Polymerization of the alkyl ether containing monomers of this invention alone, or with comonomers, may be carried out by employing initiators which generate free-radicals on application of an activating energy as is conventionally used in the polymerization of ethylenically unsaturated monomers. Included among free-radical initiators are the conventional thermally activated initiators such as azo compounds, organic peroxides and organic hydroperoxides. Representative examples of such initiators include benzoyl peroxide, tertiary-butyl perbenzoate, diisopropyl peroxydicarbonate, cumene hydroperoxide, azobis(isobutryonitrile), and the like. Generally, from about 0.01 to 5 percent by weight of thermal initiator is used.

UV-initiated polymerization is carried out using photoinitiators. Such initiators are well known and have been described, for example, in polymerization art, e.g., Chapter II of “Photochemistry” by Calvert and Pitts, John Wiley & Sons (1966). The preferred initiators are photoinitiators, which facilitate polymerization when the composition is irradiated. Representative examples of such initiators include acyloin and derivatives thereof, such as benzoin, benzoin methyl ether, benzoin ethyl ether, benzoin isopropyl ether, benzoin isobutyl ether and α-methylbenzoin; diketones such as benzil and diacetyl, etc.; ketones such as acetophenone, α,α,α-tribromoacetophenone, α,α-diethoxyacetophenone (DEAP), 2-hydroxy-2-methyl-1-phenyl-1-propanone, o-nitro-α,α,α-tribromoacetophenone, benzophenone and p,p′-tetramethyldiaminobenzophenone; α-acyloxime esters such as benzil-(O-ethoxycarbonyl)-α-monoxime; ketone/amine combinations such as benzophenone/N-methyldiethanolamine, benzophenone/tributylamine and benzophenone/Michler's ketone; and benzil ketals such as benzil dimethyl ketal, benzil diethyl ketal and 2,5-dichlorobenzil dimethyl ketal. Normally, the photoinitiator is used in amounts ranging from about 0.01 to 5% by weight of the total composition.

Visible light polymerization is carried out using initiators that are activated by visible light, especially blue light. Representative examples include ferrocenium salts, aryldiazonium salts, diaiyliodonium salts and triarylsulfonium salts, camphorquinone systems and dye/co-initiator systems.

Polymerization can be carried out in bulk in a conventional manner or in the presence of a solvent. Solvents are some times required to compatibilize components, including the drug when present. The amount of solvent depends on the nature and relative amounts of comonomers and drug, if present. Useful solvents for compatibilization include ketones, like acetone, methyl ethyl ketone, methyl propyl ketone, methyl isobutyl ketone and cyclohexane; alcohols like methanol, ethanol, isopropanol or ethyl-cellosolve; ethers like ethylene glycol or diethylene glycol dimethyl ether; esters like ethyl acetate or isopropyl acetate; dimethyl sulfoxide; N-methylpyrrolidone; N,N-dimethylformamide; N,N-dimethylacetamide and the like.

The polymerization can be carried out in molds, which can be formed of plastics, glass or metal or any other suitable material and can be any shape, for example, film, sheet or rod.

The monomer mixture can be polymerized as is, or it can be polymerized with the drug included. After the polymerization, the casting is removed from the mold and any solvent present is removed by conventional means.

In the case where the drug is not included in the polymerization mixture, a drug loading step is necessary. This is generally accomplished by dissolving the drug in an appropriate solvent (e.g., one that swells the matrix polymer) and placing the matrix polymer in that solution to allow drug uptake. Once equilibrium is reached the matrix, loaded with drug, is then removed from the solvent and dried.

Suitable drugs or active agents that can be utilized with the present delivery devices include, by way of example only, but are not limited to:
Anti-infectives: such as antibiotics, including tetracycline, chlortetracycline, bacitracin, neomycin, polymyxin B, gramicidin, oxytetracycline, chloramphenicol, and erythromycin; sulfonamides, including sulfacetamide, sulfamethizole, sulfisoxazole; quinolones, including ofloxacin, norfloxacin, ciprofloxacin, sporfloxacin; aminoglycosides, including amikacin, tobramycin, gentamicin; cephalosporins; combinations of antibiotics; antivirals, including idoxuridine, trifluridine, vidarabine cidofovir, foscarnet sodium, ganciclovir sodium and acyclovir; antifungals such as amphotericin B, nystatin, flucytosine, fluconazole, natamycin, miconazole and ketoconazole; and other anti-infectives including nitrofurazone and sodium propionate.
Antiallergenics: such as antzoline, methapyriline, chlorpheniramine, pyrilamine and prophenpyridamine, emedastine, ketorolac, levocabastin, lodoxamide, loteprednol, naphazoline/antazoline, naphazoline/pheniramine, olopatadine and cromolyn sodium.
Anti-inflammatories: such as hydrocortisone, hydrocortisone acetate, dexamethasone, dexamethasone 21-phosphate, fluocinolone, medrysone, prednisolone, prednisolone 21-phosphate, prednisolone acetate, fluorometholone, fluorometholone acetate, meddrysone, loteprednol etabonate, rimexolone.
Nonsteroidal anti-inflammatories: such as flurbiprofen, suprofen, diclofenac, indomethacin, ketoprofen, and ketorolac.
Decongestants: such as phenylephrine, naphazoline, oxymetazoline, and tetrahydrazoline.
Miotics and anticholinesterases: such as pilocarpine, eserine talicylate, carbachol, diisopropyl fluorophosphate, phospholine iodide, and demecarium bromide.
Mydriatics: such as atropine sulfate, cyclopentolate; homatropine, scopolamine, tropicamide, eucatropine, and hydroxyamphetamine.

Furthermore, the following active agents are also useful in the present devices:
Antiglaucoma agents: such as adrenergics, including epinephrine and dipivefrin, epinephryl borate; β-adrenergic blocking agents, including levobunolol, betaxolol, metipranolol, timolol, carteolol; α-adrenergic agonists, including apraclonidine, clonidine, brimonidine; parasympathomimetics, including pilocarpine, carbachol; cholinesterase inhibitors, including isofluorophate, demecarium bromide, echothiephate iodide; carbonic anhydrase inhibitors, including dichlorophenamide acetazolamide, methazolamide, dorzolamide, brinzolamide, dichlorphenamide; prostaglandins, including latanoprost, travatan, bimatoprost; diconosoids and combinations of the above, such as a β-adrenergic blocking agent with a carbonic anhydrase inhibitor.
Anticataract drugs: such as aldose reductase inhibitors including tolerestat, statol, sorbinil; antioxidants, including ascorbic acid, vitamin E; nutritional supplements, including glutathione and zinc.
Lubricants: such as glycerin, propylene glycol, polyethylene glycol and polyglycerins.

The present invention provides polymeric carriers, containing a medicinal agent, and fashioned into a medical device for the treatment of certain conditions and diseases. As such, the present invention may be described in certain embodiments as a method of treating medical disorders and diseases in a mammal comprising administering to said mammal a device containing medication to provide a controlled and sustained therapeutic effect to said mammal. An aspect of the present invention is also a method of providing continued therapy to a mammal by administering in a prescribed manner to said mammal. In certain preferred embodiments of the invention, a mammal or patient to receive the device may be a human or animal. In another preferred embodiment the device is an ocular device. In yet another preferred embodiment the ocular device as described in this invention and methods is a polymeric matrix containing a medicinal compound. As disclosed herein and as used in compositions and methods of the present invention, an ocular device may be formulated and manufactured from either a bioerodible or a non-erodible polymeric system. Effective dosages described herein include, but are not limited to, an amount of medicinal compound from about 0.01 mg to about 50.0 mg per dose delivered over a period of time in the form of an ocular device. The medicinal compound may be delivered from the ocular device of this invention in a continuous fashion over a period of days, weeks or months.

It is well known in the pharmaceutical art to prescribe medicinal agents based on whether the patient is a human or animal and based on the type and severity of the disorder or disease. The ocular devices of this invention are well within the skill of a practitioner in the art. In an alternate method of describing an effective dose, an effective amount may be described, in certain embodiments as an amount that is effective to eradicate a diseased state, such as an infection or inflammation. Alternatively, the ocular devices of this invention can be utilized to treat and control ocular disorders and diseases such as glaucoma.

In certain aspects, the present invention includes pharmaceutical ocular devices containing a medicinal agent or a combination of medicinal agents in a concentration(s) sufficient to treat or cure ocular conditions and diseases. Also in certain aspects, the present invention includes veterinary devices that can be utilized to treat ocular conditions and diseases in an animal.

An aspect of the present invention may also be described as a therapeutic package for dispensing to, or for use in dispensing to, a mammal being treated for a medical condition, disorder or disease. In the case of a device utilized to treat an ocular condition or disease the therapeutic package comprises:
(1) A medical device containing a prescribed amount of a medicinal agent packaged in a container, which is constructed from either glass or plastic. The device may be either in a sterile or a non-sterile state within the package. The dosage form contains sufficient medicinal agent that is effective to lessen, stabilize or eradicate medical conditions, disorders or diseases when administered over a defined period of time.
(2) A finished pharmaceutical container or package therefore, said container containing
   (a) a medical device containing a medicinal agent
   (b) labeling directing the use of said package in the treatment of said mammal

The compositions of this invention in the form of a medical device containing medicinal agent, for the continuous, sustained release of said medicinal agent can be packaged in an appropriate container. The physician or the patient would utilize the packaged product in accordance with the prescribed regimen. Typically, in the case of an ocular device the physician would insert the device under the upper or the lower eyelid. In other cases, the patient would insert the device under the upper or the lower eyelid. The ocular device would be maintained, in place, for the prescribed period of time. The product container and associated packaging will bear identification, information and instructions in accordance with local, federal and foreign governmental regulations. The inclusion of a “package insert” is also generally required. The “package insert” will provide information pertaining to contents, action, indications, contraindications, warning, how supplied, safety information and precautions, as well as directions for use.

The following examples are merely illustrative of the present carriers for controlled delivery of an active agent and the examples should not be considered as limiting its scope in any way.

A key to the ingredients used in Examples 1 through 10 is given in Table II.

TABLE II

| CODE | DESCRIPTION | SOURCE | CAT. NO. |
|---|---|---|---|
| DEGEMA | Di(ethyleneglycol)ethylether methacrylate | Aldrich | 41,230-9 |
| P-330 | Polyethyleneglycol(330) dimethacrylate | Aldrich | 46,980-7 |
| P-875 | Polyethyleneglycol(875) dimethacrylate | Aldrich | 43,746-8 |
| EEMA | 2-Ethoxyethyl methacrylate | Aldrich | 28,066-6 |
| TRIS | Methylacyloxypropyltris(trimethylsiloxy)silane | Gelest | SIM6487.6 |
| AZ0 | 2,2′-azobisisobutyronitrile | Aldrich | 44,109-0 |
| TFEMA | 2,2,2-Trifluoroethyl methacrylate | Sigma | 37,376-1 |
| MA | Methacrylic acid | Sigma | 39,537-4 |
| EHA | 2-Ethylhexanoic acid | Aldrich | 24-073-7 |
| TFB | Timolol Free Base | — | — |
| PRED | Prednisolone | Aldrich | P-6004 |
| TETRA | Tetracycline | Sigma | T-3258 |
| DIPYR | Dipyridamole | Sigma | D9766 |
| BROM | Brimonidine | Sigma | UK 14,304 |
| SR1129 | SarCure SR1129 | Sartomer | SR1129 |
| MEOH | Methanol | Various | — |

### Example 1

The following example details the purification of the monomers utilized in exemplary formulations for the present devices (e.g., carriers). Impurities and inhibitors are removed from the as-received monomers through adsorption onto aluminum oxide. The procedure is as follows: Approximately 2.0 gm of aluminum oxide, activated and basic, is added to a 100 ml wide mouth jar followed by addition of approximately 20.0 gm of monomer. A magnetic stir bar is added to the jar, the jar is capped, and the contents gently stirred for about two days. The purified monomer is recovered by filtration through a 0.45 micron syringe filter. The purified monomer is stored under refrigeration until use. Methacrylic acid was distilled prior to use due to its acidic nature.

### Example 2

The following procedure illustrates the formulation and polymerization of certain exemplary compositions. It should be understood that this is one of many processes that can be utilized in the practice of the present devices and should not be taken as limiting the invention.

The initiator and drug are dissolved directly in the purified monomer or monomer mix to form a clear solution. Alternatively, the initiator and drug are dissolved in an appropriate solvent and then combined with the purified monomer or monomers. The formulation is then transferred to a small test tube, usually a 10 mm×75 mm test tube. The formulation is purged with nitrogen to remove oxygen. The tube is then stoppered and placed in a 50° C. water bath and the polymerization process is allowed about three days. At that time the polymer is removed from the tube and, if present, the solvent is allowed to evaporate at room temperature for five to seven days. At that point the polymer/drug combination is ready for drug release studies.

### Example 3

The following formulations represent polymer vehicles that are useful as membranes or matrices for the controlled delivery of drugs.

| | Amount | Amount |
|---|---|---|
| Ingredient | A | B |
| DEGEMA | 100 ml | — |
| EEMA | — | 100 ml |
| AZ0 | 0.60 gm | 0.60 gm |

The monomers were purified by the procedure detailed in Example 1 and the formulations polymerized by the method given in Example 2. The resulting polymers were both clear. Sample A was flexible and Sample B was significantly stiffer.

### Example 4

The following formulations represent polymer vehicles that are useful as membranes or matrices for the controlled delivery of drugs.

| | Amount | | Amount | | Amount | | Amount | |
|---|---|---|---|---|---|---|---|---|
| Ingredient | A | | B | | C | | D | |
| DEGEMA | 80 | ml | 85 | ml | 90 | ml | 87.5 | ml |
| TFEMA | 15 | ml | 10 | ml | 5 | ml | 10 | ml |
| MA | 5 | ml | 5 | ml | 5 | ml | 2.5 | ml |
| AZ0 | 0.60 | gm | 0.60 | gm | 0.60 | gm | 0.60 | gm |

The monomers were purified by the procedure detailed in Example 1 and the formulations polymerized by the method given in Example 2. The resulting polymers were clear and the stiffness decreased from sample A to Sample D.

### Example 5

The following formulations represent a matrix drug delivery system for the controlled release of the glaucoma drug timolol.

Amount

| Ingredient | A | B | C | D |
|---|---|---|---|---|
| DEGEMA | 100 ml | 70 ml | 70 ml | 95 ml |
| TRIS | — | 30 ml | — | — |
| TFEMA | — | — | 30 ml | — |
| MA | — | — | — | 5 ml |
| AZO | 0.60 gm | 0.60 gm | 0.60 gm | 0.60 gm |
| TFB | 5.0 gm | 5.0 gm | 5.0 gm | 5.0 gm |

The monomers were purified by the procedure detailed in Example 1 and the formulations polymerized by the method given in Example 2. The resulting polymers were clear and rubbery.

### Example 6

The following formulations represent the use of different drugs in the polymeric matrix systems of this invention.

| | Amount | | |
|---|---|---|---|
| Ingredient | A | B | C |
| DEGEMA | 100 ml | 100 ml | 100 ml |
| AZO | 0.60 gm | 0.60 gm | 0.60 gm |
| PRED | 5.0 gm | — | — |
| TETRA | — | 5.0 gm | — |
| DIPYR | — | — | 5.0 gm |
| MEOH | 37.5 ml | 50 ml | 50 ml |

The monomer was purified by the procedure detailed in Example 1 and the formulations polymerized by the method given in Example 2. The resulting polymers, after drying, were rubbery; Sample A was translucent, Sample B was slightly brown and Sample C was slightly yellow.

### Example 7

The following formulations represent a matrix drug delivery system for the controlled release of the glaucoma drug timolol.

Amount

| Ingredient | A | | B | |
|---|---|---|---|---|
| DEGEMA | 87.5 | ml | 87.5 | ml |
| TFEMA | 10.0 | ml | 10.0 | ml |
| MA | 2.5 | ml | 2.5 | ml |
| AZ0 | 0.60 | gm | 0.60 | gm |
| TFB | 5.0 | gm | 2.5 | gm |

The monomers were purified by the procedure detailed in Example 1 and the formulations polymerized by the method given in Example 2. The resulting polymers were clear and flexible.

### Example 8

The following formulation represents a matrix delivery system for the controlled release of the glaucoma drug brimonidine.

| Ingredient | Amount | |
|---|---|---|
| DEGEMA | 97.5 | ml |
| MA | 2.5 | ml |
| BROM | 2.0 | gm |
| AZO | 0.60 | gm |
| MEOH | 37.5 | ml |

The monomers were purified by the procedure detailed in Example 1 and the formulations polymerized by the method given in Example 2. The resulting polymer was yellow, transparent and flexible.

### Example 9

The following example details the method utilized to monitor drug release from the polymer/drug compositions of this invention, more specifically those disclosed in Example 5 and 7.

Solutions of timolol maleate, in a concentration range of 5 ppm to 1,000 ppm, were prepared in Unisol® 4 buffer (Unisol® 4 is a preservative-free pH-balanced saline solution manufactured by Alcon Laboratories). A UV scanning spectrometer was utilized to generate a calibration curve of concentration, in gm/ml, of timolol maleate (λₘₐₓ=294) versus absorbance. A calibration curve for timolol free base was then generated.

A sample of drug loaded polymer weighing between 100 and 150 mg and of similar shape was placed in a 4 ml vial. To the vial was added 2.0 ml of Unisol® 4 buffer. After 24 hours at 37° C., the sample was removed and placed in another 4 ml vial and covered with 2.0 ml of fresh Unisol® 4 buffer. The 24-hour release vial was capped, labeled and held for analysis. This procedure was repeated four more times to obtain 1-, 2-, 3-, 4- and 5-day release data. The sampling interval was then expanded to every 3 to 5 days. The release study was carried out for a total of up to 90 days.

The drug release samples were analyzed by UV spectroscopy and absorbance readings converted to weight of drug via the calibration curve. A plot of cumulative weight of drug released versus time was generated.

### Example 10

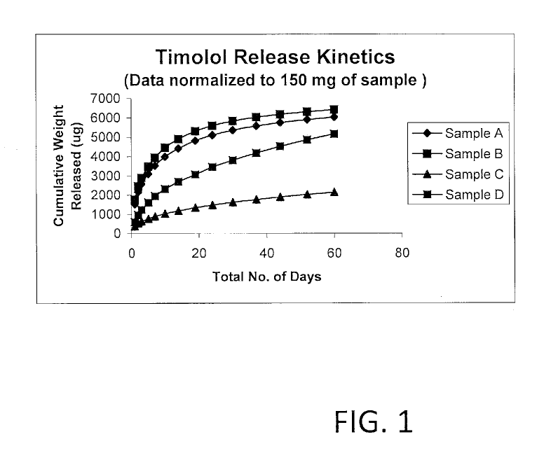

The following example illustrates the controlled release of timolol from the polymeric matrices described in Example 5. The timolol release characteristics of the polymeric matrices described in Example 5 were determined by the methodology established in Example 9. The cumulative release, in micrograms, was plotted against elapsed time in days. The results were normalized to 0.150 gm of sample weight for comparison purpose and are shown in FIG. 1.

The homopolymer DEGEMA and the copolymer of DEGEMA and TRIS exhibited about the same release kinetics. Timolol was released rather rapidly over a 20 to 30 day period. The copolymer of DEGEMA and methacrylic acid provided a slower release of timolol over a 30 to 40 day period. The copolymer of DEGEMA and the fluoromonomer TFEMA provided a relatively constant release of timolol from about 5 days to 60 days.

### Example 11

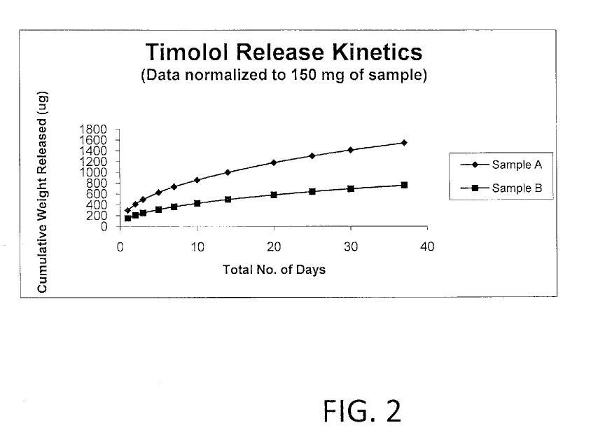

The following example illustrates the controlled release of timolol from the polymeric matrices described in Example 7. The timolol release characteristics of the polymeric matrices described in Example 7 were determined by the methodology established in Example 9. The cumulative release, in micrograms, was plotted against elapsed time in days. The results were normalized to 0.150 gm of sample weight for comparison purpose and are shown in FIG. 2.

Both samples A and B exhibit relatively constant and slow release of timolol from about 5 days to about 40 days. The doubling of the concentration of timolol, sample A, provides a predictable doubling of the amount of timolol released over time.

### Example 12

The formulations of this invention can be photo polymerized using methods known in the art. Polymerizations are carried out in a UV curing chamber such as Model CL-1000L available from UV Process Supply, Inc. This chamber operates at a UV wavelength of 365 nm and can provide a maximum UV energy exposure setting of 999,900 micro joules per cm². Both UV energy exposure and time of exposure can be varied to maximize polymerization efficiency. Formulations containing a UV initiator are placed in a vial then purged with nitrogen to remove oxygen. The vials are quickly stoppered to exclude reintroduction of oxygen. The stoppered vial of formulation is placed in a glove box along with two piece polypropylene mold halves. The glove box is then purged with nitrogen to remove oxygen. Once this has been accomplished the formulation is opened and a prescribed amount of formulation is pipetted into the base half of the polypropylene mold. The second mold half, the cover, is fitted into the mold base to seal off the formulation and form the desired device geometry. The filled molds are then placed into the UV chamber and exposed to a prescribed energy level for a prescribed amount of time. The polymerized devices are then removed for the molds.

### Example 13

The following formulations represent polymer vehicles that are useful as membranes or matrices for the controlled delivery of drugs.

| | Amount | | | | | | | | | |
|---|---|---|---|---|---|---|---|---|---|---|
| Ingredient | A | B | C | D | E | F | G | H | I | J |
| DEGEMA | 100 ml | 95.0 ml | 95.0 ml | 70.0 ml | | 99.0 ml | 95.0 ml | 65.0 ml | 75.0 ml | 80.0 ml |
| TFEMA | | | | 30.0 ml | 30.0 ml | | | 30.0 ml | 20.0 ml | 15.0 ml |
| P-330 | | 5.0 ml | | | | | 5.0 ml | | 5.0 ml | 5.0 ml |
| P-875 | | | 5.0 ml | | 70.0 ml | | | 5.0 ml | | |
| MA | | | | | | 1.0 ml | 1.0 ml | 1.0 ml | 1.0 ml | 1.0 ml |
| SR1129 | 0.40 gm | 0.40 gm | 0.40 gm | 0.40 gm | 0.40 gm | 0.40 gm | 0.40 gm | 0.40 gm | 0.40 gm | 0.40 gm |

The monomers were purified by the procedure detailed in Example 1 and the formulations polymerized by the method given in Example 12. The UV exposure energy was 120,000 micro joules per cm² and the exposure time was 30 minutes. The resulting polymers were clear and exhibited varying degree of flexibility.

### Example 14

The following formulations represent a matrix drug delivery system for the controlled release of the glaucoma drug timolol.

Amount

| Ingredient | A | | B | |
|---|---|---|---|---|
| DEGEMA | 65.0 | ml | 65.0 | ml |
| TFEMA | 30.0 | ml | 30.0 | ml |
| P-330 | 5.0 | ml | 5.0 | ml |
| MA | | | 1.0 | ml |
| SR1129 | 0.13 | gm | 0.13 | gm |
| TFB | 5.0 | gm | 5.0 | gm |

The monomers were purified by the procedure detailed in Example 1 and the formulations polymerized by the method given in Example 12. The UV exposure energy was 60,000 micro joules per cm² and the exposure time was 30 minutes. The resulting polymers were clear and flexible.

### Example 15

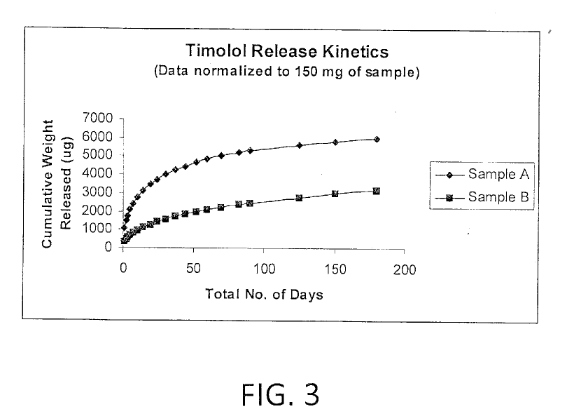

The following example illustrates the controlled release of timolol from the polymeric matrices described in Example 14. The timolol release characteristics of the polymeric matrices described in Example 14 were determined by the methodology established in Example 9. The cumulative release, in micrograms, was plotted against elapsed time in days. The results were normalized to 0.150 gm of sample weight for comparison purpose and are shown in FIG. 3.

The results demonstrate the ability of an acidic monomer, in this case methacrylic acid to dramatically decrease the release rate of the timolol. This is the result of the formation of an acid/base complex within the polymer matrix, which significantly reduces the release rate of the timolol. This concept can be applied to other drugs whether acidic or basic by inclusion of either an acidic or basic monomer within the polymer matrix to form the acid/base complex.

### Example 16

The following formulation represents a matrix drug delivery system for the controlled release of the glaucoma drug timolol.

| Ingredient | Amount | |
|---|---|---|
| DEGEMA | 65.0 | ml |
| TFEMA | 30.0 | ml |
| P-330 | 5.0 | ml |
| EHA | 1.0 | ml |
| SR1129 | 0.40 | gm |
| TFB | 5.0 | gm |

The 2-ethylhexanoic acid (EHA) was used as received. The monomers were purified by the procedure detailed in Example 1 and the formulation polymerized by the method given in Example 12. The UV exposure energy was 120,000 micro joules per cm² and the exposure time was 30 minutes. The resulting polymer was clear and flexible.

This example illustrates the use of an organic acid component to form an internal acid/base complex with the timolol. In addition, the 2-ethylhexanoic acid functions as a permeability enhancer for the drug timolol.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing form the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-22. (canceled)

23. An ocular device comprising:
a carrier for controlled delivery of an active agent, the carrier comprising:
a polymeric matrix including an alkyl ether derived from at least one monomer having a formula:
P-Y-O—(CH₂)ₓ—[O—(CH₂)_{y}]ₙ—O-T
wherein:
P is an ethylenically unsaturated polymerizable group selected from the group consisting of:
CH₂═CH—
and 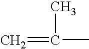
and Y is a spacer group selected from the group consisting of:
—CO—
—OCO—
—CONHCH₂—
—CONHCH₂CH₂CH₂—
—COOCH₂CH₂NHCOCH₂—
—COOCH₂CH₂NHCH₂CH(OH)CH₂—
—CH₂—
—CH₂CH₂—
—CH₂CH₂CH₂—
—CH₂CH₂CH₂CH₂—
—C₆H₄—
—C₆H₄CH₂—
—COOCH₂CH(OH)CH₂—
—COOCH₂CH₂—
—COOCH₂CH₂OCH₂CH₂— and
—COOCH₂CH₂NHCO—
T is a terminal group comprising an alkyl group or a P-Y group
x is an integer from 2 to about 6
y is an integer from 2 to about 8
n is an integer 0 to about 50; and
wherein the carrier is configured in the shape of the ocular device.

24. The ocular device of claim 23, wherein the contained active agent is the anti-glaucoma drug timolol.
